(19) United States
(12) Patent Application Publication
(10) Pub. No.: US 2024/0407838 A1
(43) Pub. Date: Dec. 12, 2024

(54) HEAT SHRINK LINE MARKER

(71) Applicant: Boston Scientific Medical Device Limited, Galway (IE)

(72) Inventors: Brock Miller, Toronto (CA); Kai-Lon Fok, Mississauga (CA); Matthew Gravett, Milton (CA)

(21) Appl. No.: 18/737,632

(22) Filed: Jun. 7, 2024

Related U.S. Application Data
(60) Provisional application No. 63/507,319, filed on Jun. 9, 2023.

(51) Int. Cl. A61B 18/14; A61B 17/00; A61B 18/00; A61B 90/92; A61M 25/09

(52) U.S. Cl. CPC A61B 18/1492; A61B 90/92; A61B 2017/00247; A61B 2018/00083; A61M 2025/09183

(57) ABSTRACT

A radiofrequency guidewire is disclosed. The system includes a core wire having a tapered section, a first insulator portion having a first insulator property and a first insulator length, and a second insulator portion having a second insulator property and a second insulator length. The first insulator portion and the second insulator portion are positioned along the core wire so as to form an overlap portion aligned with the tapered section of the core wire.

### CROSS REFERENCE TO RELATED APPLICATIONS

The present application claims priority to U.S. Provisional Patent Application Ser. No. 63/507,319, filed Jun. 9, 2023, the entire disclosure of which is incorporated herein by reference.

### TECHNICAL FIELD

The present invention relates generally to methods and devices for a radiofrequency guidewire. More specifically, the present invention is concerned with visual line markers on a radiofrequency guidewire.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein and directed towards the heart. This device relies on the use of mechanical force to drive the sharp tip through the septum. Alternatively, devices currently exist for access to the epicardial space. Access to the space may be initiated with a mechanical puncture device using a large bore needle, like a Tuohy-style needle.

In certain applications, a medical guidewire must be lined up with another device. In these guidewire alignment applications, it is desirable to have a visual line marker on the wire that will align with a marker on the device with which the wire is intended to line up with. These devices may include the back of a hub, a hemostasis valve, among others. This enables a user to visually align the guidewire relative to the external device. Guidewires currently on the market use ink line markers that are printed onto the guidewire. These markings generally wipe or scrape off the guidewire during normal use, making it difficult for the user to line up the guidewire with other devices and proceed with the procedure.

Against this background, there exists a continuing need in the industry to provide improved visual line marker guidewire devices and methods. An object of the present invention is therefore to provide such improved guidewires.

### SUMMARY

In Example 1, a radiofrequency guidewire includes a core wire having a tapered section, a first insulator portion having a first insulator property and a first insulator length, and a second insulator portion having a second insulator property and a second insulator length; wherein the first insulator portion and the second insulator portion are positioned along the core wire so as to form an overlap portion aligned with the tapered section of the core wire.

Example 2 is the radiofrequency guidewire of Example 1 wherein the first insulator portion is made of a heat shrink material.

Example 3 is the radiofrequency guidewire of Example 1 wherein the second insulator portion is made of a heat shrink material

Example 4 is the radiofrequency guidewire of Example 1 wherein the first insulator property is made of one color.

Example 5 is the radiofrequency guidewire of any of Examples 1-4 wherein the second insulator property is made of a different color than the first insulator property color.

Example 6 is the radiofrequency guidewire of any of Examples 1-5 wherein the color change of the first insulator portion and the second insulator portion creates a visual marker on the core wire.

Example 7 is the radiofrequency guidewire of Example 1 wherein the first insulator length and the second insulator length define an overall core wire length.

Example 8 is the radiofrequency guidewire of Example 1 wherein the first insulator portion and the second insulator portion are overlapped to maintain a constant outer diameter.

Example 9 is the radiofrequency guidewire of Example 1 wherein the overlap portion is insulated.

Example 10 is the radiofrequency guidewire of Example 1 wherein the first insulator portion and the second insulator portion are butted and recovered together at the tapered section of the core wire.

Example 11 is the radiofrequency guidewire of any of Examples 1-10 wherein the overlap portion includes an orientation in which the first insulator portion is on top of the second insulator portion at the tapered section of the core wire.

Example 12 is the radiofrequency guidewire of any of Examples 1-11 wherein the heat shrink material of the first insulator portion and the second insulator portion is made of any electrically insulative and lubricious thermoplastic.

Example 13 is the radiofrequency guidewire of Example 1 wherein the guidewire is inserted into an external device.

Example 14 is the radiofrequency guidewire of any of Examples 1-13 wherein the first insulator portion is inserted into the external device first.

Example 15 is the radiofrequency guidewire of Example 1 wherein the guidewire may be a radiofrequency perforation device.

In Example 16, a radiofrequency guidewire includes a core wire having a tapered section, a first insulator portion having a first insulator property and a first insulator length, and a second insulator portion having a second insulator property and a second insulator length; wherein the first insulator portion and the second insulator portion are positioned along the core wire so as to form an overlap portion aligned with the tapered section of the core wire.

Example 17 is the radiofrequency guidewire of Example 16 wherein the first insulator portion and the second insulator portion are made of a heat shrink material.

Example 18 is the radiofrequency guidewire of Example 16 wherein the first insulator property is made of one color.

Example 19 is the radiofrequency guidewire of Example 18 wherein the second insulator property is made of a different color than the first insulator property color.

Example 20 is the radiofrequency guidewire of Example 19 wherein the color change of the first insulator portion and the second insulator portion creates a visual marker on the core wire.

Example 21 is the radiofrequency guidewire of Example 16 wherein the first insulator length and the second insulator length define an overall core wire length.

Example 22 is the radiofrequency guidewire of Example 16 wherein the first insulator portion and the second insulator portion are overlapped to maintain a constant outer diameter.

Example 23 is the radiofrequency guidewire of Example 16 wherein the overlap portion is insulated.

Example 24 is the radiofrequency guidewire of Example 16 wherein the first insulator portion and the second insulator portion are butted and recovered together at the tapered section of the core wire.

Example 25 is the radiofrequency guidewire of Example 16 wherein the overlap portion includes an orientation in which the first insulator portion is on top of the second insulator portion at the tapered section of the core wire.

In Example 26, a radiofrequency guidewire includes a core wire having a tapered section, a first insulator portion having a first insulator property and a first insulator length, and a second insulator portion having a second insulator property and a second insulator length; wherein the first insulator portion and the second insulator portion are positioned along the core wire so as to form an overlap portion aligned with the tapered section of the core wire, and wherein the first insulator portion and the second insulator portion are made of a heat shrink material.

Example 27 is the radiofrequency guidewire of Example 26 wherein the first insulator property is made of one color.

Example 28 is the radiofrequency guidewire of Example 27 wherein the second insulator property is made of a different color than the first insulator property color.

Example 29 is the radiofrequency guidewire of Example 28 wherein the color change of the first insulator portion and the second insulator portion creates a visual marker on the core wire.

Example 30 is the radiofrequency guidewire of Example 26 wherein the first insulator length and the second insulator length define an overall core wire length.

Example 31 is the radiofrequency guidewire of Example 26 wherein the first insulator portion and the second insulator portion are overlapped to maintain a constant outer diameter.

Example 32 is the radiofrequency guidewire of Example 26 wherein the overlap portion is insulated.

Example 33 is the radiofrequency guidewire of Example 26 wherein the overlap portion includes an orientation in which the first insulator portion is on top of the second insulator portion at the tapered section of the core wire.

Example 34 is the radiofrequency guidewire of Example 26 wherein the guidewire is inserted into an external device.

In Example 35, a method of making a radiofrequency guidewire includes providing a core wire having a tapered section. The method of making a radiofrequency guidewire also includes securing a first insulator portion having a first insulator property and a first insulator length. The method of making a radiofrequency guidewire further includes securing a second insulator portion having a second insulator property and a second insulator length; wherein the first insulator portion and the second insulator portion are positioned along the core wire so as to form an overlap portion aligned with the tapered section of the core wire.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A and 1B are schematic illustrations of a medical procedure within a patient's heart for gaining access to the transseptal and epicardial space, according to embodiments of the present disclosure.

FIGS. 2A-2G are schematic and cross-sectional illustrations of a core wire having a first insulator portion and a second insulator portion positioned along the core wire, according to an embodiment of the present disclosure.

FIGS. 3A and 3B are cross-sectional illustrations of an overlap portion orientation of the core wire of FIGS. 2A-2G, according to an embodiment of the present disclosure.

FIGS. 4A and 4B are schematic illustrations of a guidewire and guidewire marker, according to an embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

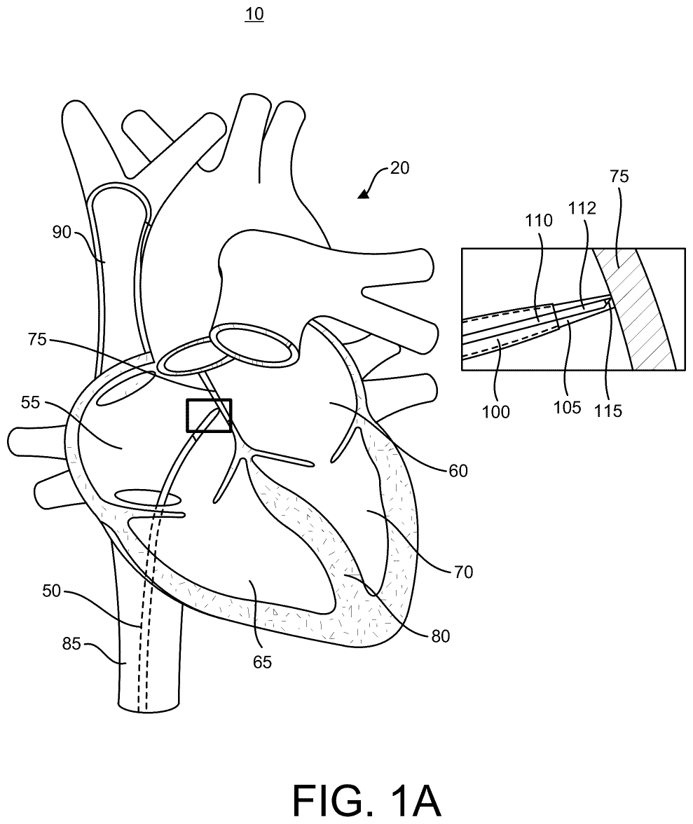

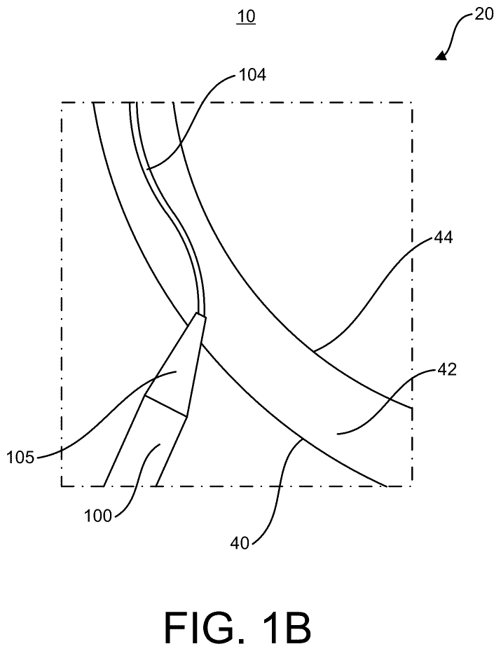

FIGS. 1A and 1B are schematic illustrations of a medical procedure within a patient's heart for gaining access to the transseptal and epicardial space, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a radiofrequency perforation device 110 having distal end portion 112 terminating in a tip electrode 115, and a guidewire. In one embodiment, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator may then be introduced into the sheath and over the guidewire, and advanced through the sheath into the SVC. Alternatively, the dilator may be fully inserted into the sheath prior to entering the body, and both may be advanced simultaneously towards the heart.

When the guidewire, sheath 100 and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. The RF perforation device 110 is then positioned such that the tip electrode 115 is aligned with or protruding slightly from the distal end of the dilator 105. With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

As shown in FIG. 1B, still another medical procedure 10 developed for diagnosing or treating physiological ailments originating within the heart 20 includes epicardial ablation to help restore a regular heart rhythm. As illustrated, the heart includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart enclosed between the two opposing serosal surfaces, often referred to as the pericardial space or pericardial cavity 42.

In embodiments, the pericardium 40 may be punctured with a needle. Once punctured, a dilator 105 is advanced to dilate the puncture created by the needle through the pericardium 40. In embodiments, a sheath 100 may be advanced with the dilator 105. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

As described above, guidewires play an important role in transseptal access systems and epicardial ablation procedures. As will be explained in greater detail herein, the present disclosure describes novel devices and methods for providing safe and efficient access to the heart using an insulator portion color change visual marker to enable the user to visually align a wire with an external marker of an external device, for example an introducer sheath.

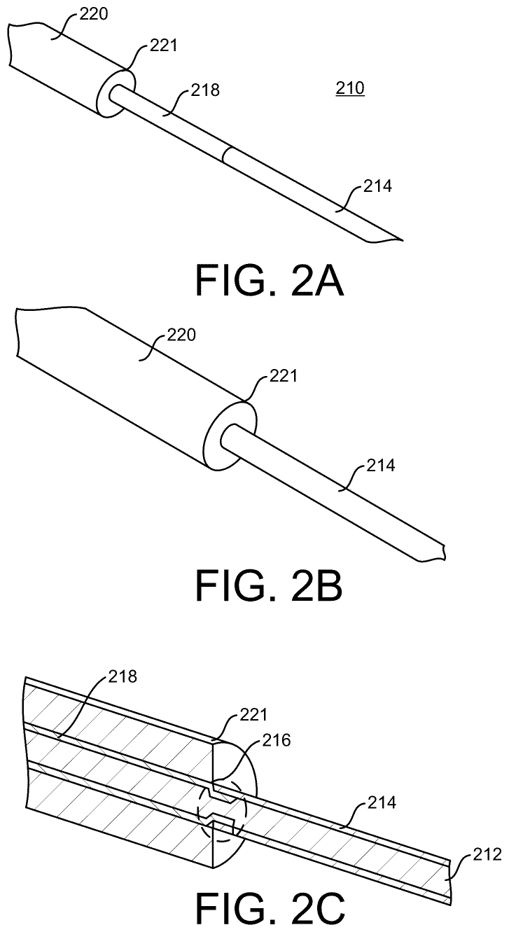

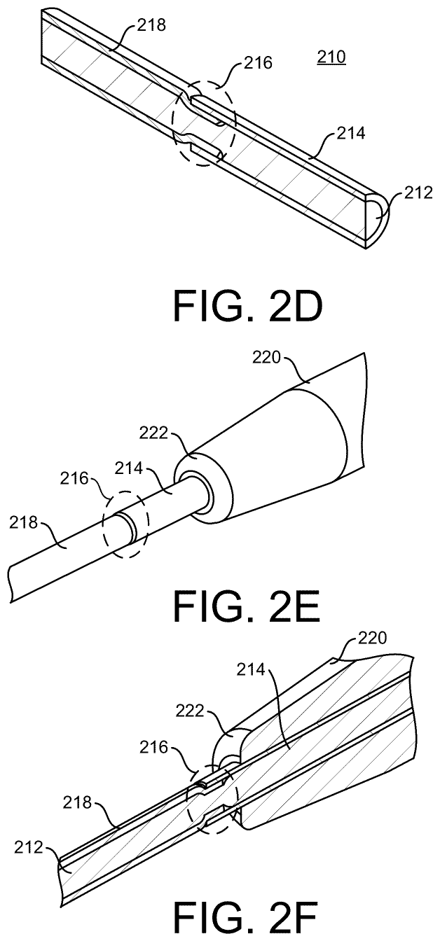

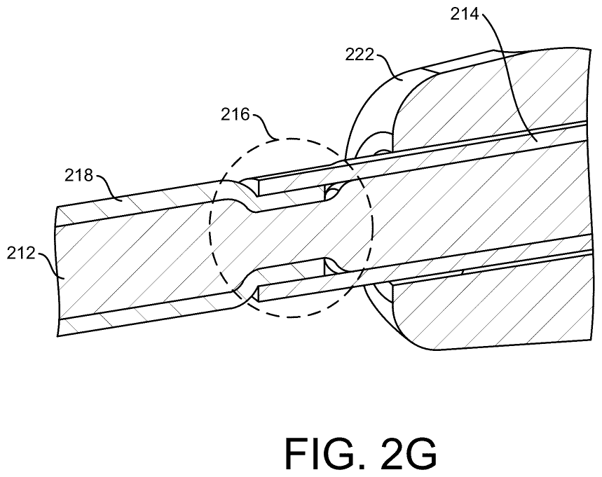

FIGS. 2A-2G are schematic and cross-sectional illustrations of a guidewire 210 having a core wire 212 with a first insulator portion 214 and a second insulator portion 218 positioned along the core wire 212, according to embodiments of the present disclosure. As shown in FIGS. 2A-2G, the core wire 212 includes a tapered section 216 that is positioned along the core wire 212. In embodiments, the core wire 212 may be a nickel-titanium (NiTi) wire. In embodiments, the guidewire 210 is a radiofrequency guidewire, which includes an electrically active tip portion. As shown, the first insulator portion 214 includes a first insulator property and a first insulator length. The second insulator portion 218 includes a second insulator property and a second insulator length. In embodiments, the first insulator portion 214 and the second insulator portion 218 are made of a heat shrink material. In embodiments, the heat shrink may include a lubricious coating to enhance insertion and retraction of the guidewire 210 within an external device. In further embodiments, the first insulator property of the first insulator portion 214 is of a first color, while the second insulator property of the second insulator portion 218 is a second color, which is different from the first color. In embodiments, the first color of the first insulator portion 214 is visually contrasting from the second color of the second insulator portion 218 so as to provide the user with optimum visibility of the color change line.

The change in color between the first insulator portion 214 and the second insulator portion 218 creates a visual marker (e.g., a visible line) on the guidewire 210, which may assist the user in aligning the guidewire 210 with the external marker on an external device 220. In embodiments, the first insulator portion 214 and the second insulator portion 218 may be coupled together to create the marker (e.g., a color-change line) that is visible on the guidewire 210. According to various embodiments, the first insulator portion 214 and the second insulator portion 218 may be for an end-to-end contact (e.g., a butt-joint) or may overlap each other. The two portions may then be connected using any variety of know techniques, including, for example, adhesive bonding, melt bonding, heat shrink or a mechanical fastener.

As shown in FIG. 2D, the tapered section 216 of the core wire 212 may result in a small gap between the portions 214 and 218, which could result in the user being exposed to electrical current in the core wire 212. Therefore, in embodiments, when joined together, the first insulator portion 214 and the second insulator portion 218 are positioned along the core wire 212 so as to form an overlap portion that is aligned with the tapered section 216 of the core wire 212. The overlapping configuration created by the first and the second insulator portions, made of heat shrink material, at the tapered section 216 allows the core wire 212 to be electrically insulated while also having a heat shrink color change to identify the line marking. Thus, the overlap portion created by the first and the second insulator portions maintains a constant outer diameter of the guidewire 210.

As further shown in FIGS. 2A-2G, the guidewire 210 may be inserted into the external device 220 and advanced longitudinally within the device. The external device 220 includes a proximal end 221 and an opposite distal end 222. In embodiments, the external device may include any device typically used with guidewires, including, for example, a guide catheter, a sheath or a dilator. The proximal end of such devices will typically include a hub or a hemostasis valve. To facilitate longitudinal alignment of the guidewire 210 with the external device 220, the guidewire 210 may be inserted into the external device 220 from the proximal end 221 of the external device 220, as shown in FIGS. 2A-2C. In embodiments, the visual marker (e.g., the visible line created by the overlap of the first and the second insulator portions) on the guidewire 210 may be aligned with the hub or hemostasis valve of the external device 220 to indicate to the user a desired position of the guidewire 210 within an outer marker of the external device. Thus, in embodiments, for example when the visual marker is aligned with the hub or hemostasis valve of the external device 220 this indicates that the distal end of the guidewire 210 extends a desired amount from the distal end of the outer guide.

In embodiments, the first insulator portion 214 and the second insulator portion 218 are made of a thermoplastic heat shrink material with electrically insulative and lubricious properties. In further embodiments, the first insulator portion 214 and the second insulator portion 218 are made of polytetrafluoroethylene (PTFE), which does not bond well with inks or dyes. This is advantageous because the heat shrink line marker on the guidewire 210 will not wipe or scrape off during use. In embodiments, in the overlap portion of the guidewire 210, a PTFE-PTFE overlap may be used in which the heat shrink of the first insulator portion 214 and the heat shrink of the second insulator portion 218 are bonded together to electrically insulate the core wire 212. Alternatively, in embodiments, a small piece of fluorinated ethylene propylene (FEP) may be used to flow between the PTFE heat shrink layers of the first and the second insulator portions to help bond the insulator portion and electrically isolate the core wire 212. In yet another embodiment, one insulator portion may be made of PTFE heat shrink while the other may be made of FEP heat shrink.

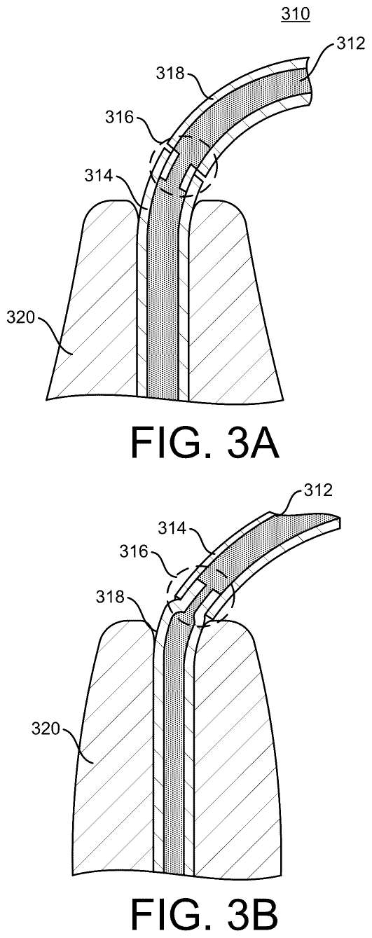

FIGS. 3A-3B are cross-sectional illustrations of different overlap orientations of a guidewire 310 having a core wire 312, according to embodiments of the present disclosure. The guidewire 310 is structurally and functionally similar to the guidewire 210 of FIGS. 2A-2G except as described herein. As shown, the core wire 312 includes a first insulator portion 314 and a second insulator portion 318 positioned along the core wire 312. As shown, the core wire 312 includes a tapered section 316 that is positioned along the core wire 312. As shown, the tapered section of the core wire 312 creates small gaps in the core wire, which could result in the user being electrically shocked. Therefore, in embodiments, the first insulator portion 314 and the second insulator portion 318 are butted and recovered together in an orientation in which the first insulator portion 314 is on top of the second insulator portion 318 so as to form an overlap portion that is aligned with the tapered section 316 of the core wire 312. In embodiments, the first insulator portion 314 and the second insulator portion 318 are made of a thermoplastic heat shrink material with electrically insulative and lubricious properties. In further embodiments, the first insulator portion 314 and the second insulator portion 318 are made of polytetrafluoroethylene (PTFE).

In certain embodiments, as shown in FIGS. 3A and 3B, the direction of the overlap is helpful in avoiding damaging the overlap portion of the first and the second insulator portions 314, 318 since the guidewire 310 is inserted and retracted through an introducer of an external device 320. In embodiments, in a routine scenario, when the guidewire 310 is deployed in an orientation in which it curves away from the main axis of the external device 320, when the guidewire 310 needs to be retracted through the external device 320, the core wire 312 and the overlapping portion of the first and the second insulator portions may slide along the tip of the external device 320. In embodiments, in a case in which the second insulator portion 318 is on top of the first insulator portion 314 at the tapered section 316, as shown in FIG. 3B, the introducer tip opening of the external device will likely get caught on the overlap portion, resulting in the tearing of the first and/or the second insulator portion. The torn insulator portions could end up as a loose body in a patient's abdominal cavity or their circulatory system. The torn insulator portions could also effectively increase the outer diameter of the guidewire, preventing it from being retracted from the introducer and therefore require the wire to be removed from the patient. Thus, it is crucial that the first insulator portion 314 and the second insulator portion 318 are butted and recovered together in an orientation in which the first insulator portion 314 is on top of the second insulator portion 318, as shown in FIG. 3A, so as to form an overlap portion that is aligned with the tapered section 316 of the core wire 312.

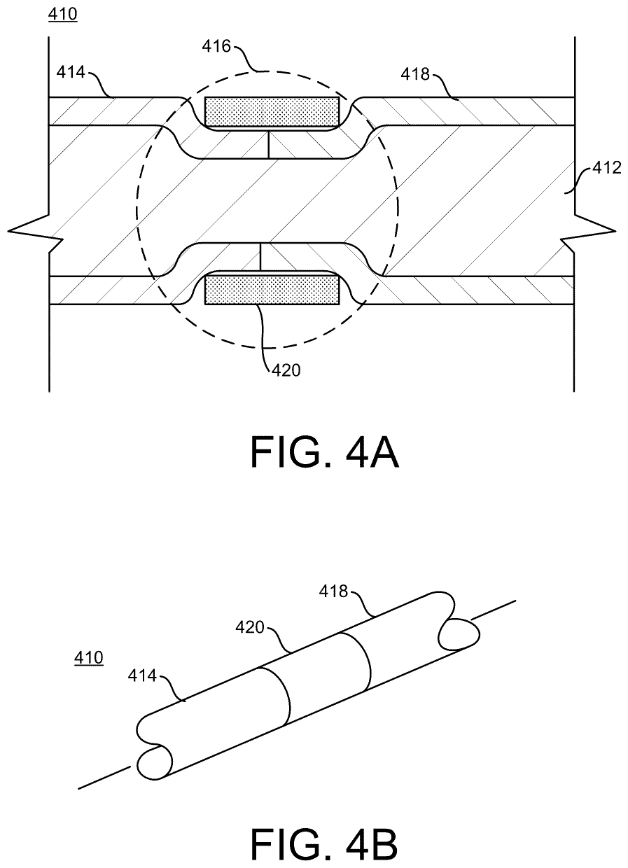

FIGS. 4A and 4B are schematic illustrations of a guidewire 410 with a guidewire marker 420, according to embodiments of the present disclosure. As shown, the guidewire 410 includes a core wire 412. As shown, a first insulator portion 414 and a second insulator portion 418 are positioned along the core wire 412. As shown, the core wire 412 includes a tapered section 416 that is positioned along the core wire. In embodiments, the tapered section 416 of the core wire 412 creates small gaps in the core wire, which could result in the user being electrically shocked. Therefore, in embodiments, the first insulator portion 414 and the second insulator portion 418 are bonded together to electrically insulate the core wire 412. As shown, the guidewire marker 420 is placed on top of the first and second insulator portions 414, 418 at the tapered section 416. In embodiments, as shown in FIG. 4B, the guidewire marker 420 is of a ring material (e.g., metal for luster, plastic for color) placed around the tapered section 416. In embodiments, the marker 420 is placed in the groove created by the tapered section 416 to ensure that the marker 420 does not move.

In embodiments, the guidewire marker 420 is made of a color different from the first insulator portion 414 and the second insulator portion 418 so as to be visually contrasting to the user. In embodiments, the marker 420 is made of a thin layer of color material, such as polytetrafluoroethylene (PTFE), which is encapsulated by a thicker layer of heat shrink material. In embodiments, the overlapping configuration created by the first and the second insulator portions 414, 418 and the guidewire marker 420, made of heat shrink material, at the tapered section 416 allows the core wire 412 to be electrically insulated while also having a heat shrink color change to identify the line marking. Thus, the overlap portion created by the first and the second insulator portions maintains a constant outer diameter of the guidewire 410.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A radiofrequency guidewire comprising:
a core wire having a tapered section;
a first insulator portion having a first insulator property and a first insulator length, and
a second insulator portion having a second insulator property and a second insulator length;
wherein the first insulator portion and the second insulator portion are positioned along the core wire so as to form an overlap portion aligned with the tapered section of the core wire.

2. The radiofrequency guidewire of claim 1, wherein the first insulator portion and the second insulator portion are made of a heat shrink material.

3. The radiofrequency guidewire of claim 1, wherein the first insulator property is made of one color.

4. The radiofrequency guidewire of claim 3, wherein the second insulator property is made of a different color than the first insulator property color.

5. The radiofrequency guidewire of claim 4, wherein the color change of the first insulator portion and the second insulator portion creates a visual marker on the core wire.

6. The radiofrequency guidewire of claim 1, wherein the first insulator length and the second insulator length define an overall core wire length.

7. The radiofrequency guidewire of claim 1, wherein the first insulator portion and the second insulator portion are overlapped to maintain a constant outer diameter.

8. The radiofrequency guidewire of claim 1, wherein the overlap portion is insulated.

9. The radiofrequency guidewire of claim 1, wherein the first insulator portion and the second insulator portion are butted and recovered together at the tapered section of the core wire.

10. The radiofrequency guidewire of claim 1, wherein the overlap portion includes an orientation in which the first insulator portion is on top of the second insulator portion at the tapered section of the core wire.

11. A radiofrequency guidewire comprising:
a core wire having a tapered section;
a first insulator portion having a first insulator property and a first insulator length, and
a second insulator portion having a second insulator property and a second insulator length;
wherein the first insulator portion and the second insulator portion are positioned along the core wire so as to form an overlap portion aligned with the tapered section of the core wire, and
wherein the first insulator portion and the second insulator portion are made of a heat shrink material.

12. The radiofrequency guidewire of claim 1, wherein the first insulator property is made of one color.

13. The radiofrequency guidewire of claim 12, wherein the second insulator property is made of a different color than the first insulator property color.

14. The radiofrequency guidewire of claim 13, wherein the color change of the first insulator portion and the second insulator portion creates a visual marker on the core wire.

15. The radiofrequency guidewire of claim 11, wherein the first insulator length and the second insulator length define an overall core wire length.

16. The radiofrequency guidewire of claim 11, wherein the first insulator portion and the second insulator portion are overlapped to maintain a constant outer diameter.

17. The radiofrequency guidewire of claim 11, wherein the overlap portion is insulated.

18. The radiofrequency guidewire of claim 11, wherein the overlap portion includes an orientation in which the first insulator portion is on top of the second insulator portion at the tapered section of the core wire.

19. The radiofrequency guidewire of claim 11, wherein the guidewire is inserted into an external device.

20. A method of making a radiofrequency guidewire, the method comprising:
providing a core wire having a tapered section;
securing a first insulator portion having a first insulator property and a first insulator length, and
securing a second insulator portion having a second insulator property and a second insulator length;
wherein the first insulator portion and the second insulator portion are positioned along the core wire so as to form an overlap portion aligned with the tapered section of the core wire.
